(19) United States
(12) Patent Application Publication
(10) Pub. No.: US 2008/0081963 A1
(43) Pub. Date: Apr. 3, 2008

(54) Methods and Apparatus for Profiling Cardiovascular Vulnerability to Mental Stress

(75) Inventors: Morteza Naghavi, Houston, TX (US); Timothy J. O'Brien, Anoka, MN (US); Craig Jamieson, Houston, TX (US); Mark C. Johnson, Houston, TX (US); Haider A. Hassan, Houston, TX (US)

Correspondence Address: WONG, CABELLO, LUTSCH, RUTHERFORD & BRUCCULERI,;L.L.P., 20333 SH 249, SUITE 600, HOUSTON, TX, 77070, US

(73) Assignee: ENDOTHELIX, INC., Houston, TX (US)

(21) Appl. No.: 11/863,490

(22) Filed: Sep. 28, 2007

Related U.S. Application Data
(60) Provisional application No. 60/827,518, filed on Sep. 29, 2006.

(51) Int. Cl. A61B 5/00

(52) U.S. Cl. 600/301

(57) ABSTRACT

Methods and apparatus are provided for determining individual responses to induced and actual mental stress and for identifying individuals susceptible to detrimental effects of mental stress on the cardiovascular system. The subjects may be at risk for chronic effects of mental stress by virtue of synergistic effects of mental stress and cardiovascular (CV) disease risk factors) or may be at risk, or vulnerable to, acute effects of mental stress by virtue of synergistic effects of mental stress and underlying hidden coronary atherosclerosis. The invention further provides methods and apparatus for assessing vascular reactivity in individuals under ambulatory conditions and relating mental stress responses to vascular reactivity.

### CROSS REFERENCE TO RELATED APPLICATIONS

This application claims priority under 35 USC §119 to U.S. Provisional Application No. 60/827,518, filed Sep. 29, 2006, the disclosure of which is incorporated herein by reference in its entirety.

### FIELD OF THE INVENTION

This invention relates to methods and apparatus for assessing both mental stress and vascular reactivity status to identify individuals vulnerable to detrimental effects of mental stress on the cardiovascular system.

### BACKGROUND OF THE INVENTION

Without limiting the scope of the invention, its background is described in connection with the interaction of psychosomatic mental stress and cardiovascular disease. Cardiovascular disease (CVD), including coronary heart disease (CHD), is the leading cause of death in the United States and in most developed countries. Non-fatal manifestations of CVD require expensive hospitalization and treatment. It is now believed that psychological factors, i.e. emotions such as depression, anger/hostility, and anxiety/mental stress, are significant both in the advancement of CVD and in impairing the measurement of disease using conventional methods. For example, depression alone may increase risk of mortality four-fold, thus having a similar prognostic value as does left ventricular dysfunction and prior history of myocardial infarction. Hostility has been associated with greater mortality rates through a specific influence on atherosclerosis, re-stenosis, and ischemia. See Mittleman M A, et al. “Triggering of acute myocardial infarction onset by episodes of anger. Determinants of Myocardial Infarction Onset Study Investigators” Circulation 92(7) (1995) 1720-1725.

Work-related mental stress exacts a tremendous toll on the U.S. population. Work-related mental stress and mental illness costs the American economy roughly $150 billion per year in lost productivity and disability claims. Contemporary medical research is becoming increasingly concerned with the significance and prevalence of psychological mental stress in modern western society. It is being increasingly recognized that mental stress—and the associated emotions of anxiety, depression, and chronic anger—lead not only to a vastly diminished quality of life, but also negatively impact the physiological health of patients either directly or indirectly. For example, psychological mental stress has been implicated by recent research in promoting a variety of pathologies including CVD. See Robinson E L, et al. “The effects of physical and mental stress on cardiovascular reactivity in a group of African American female college students” Journal of Anxiety Disorders 10 (6) (1996) 543-553.

In another study on the effects of biofeedback and relaxation on patients with diabetes, it was found that those patients who suffered from depression retained higher levels of blood glucose. McGinnis R A, et al. “Biofeedback-assisted relaxation in type 2 diabetes” Diabetes Care 28 (9) (September 2005) 2145-2149. When combined with associated or independent poor behavioral or life-style factors (overindulgence or poor diet, lack of exercise, lack of sleep, smoking, drug usage), mental stress forms an important link in the causal chain (or cycle) that leads to deteriorating health and quality of life. The causal pathways from psychological factors to CVD may be direct as well as indirect. Depression results in dysregulation of autonomic nervous system functioning, leading to impaired platelet functioning, elevated heart rate, reduced heart rate variability (HRV), and impaired vagal control. See Carney Met al., “Change in heart rate and heart rate variability during treatment for depression in patients with coronary heart disease” Psychosomatic Medicine 62 (2000) 639-47. Anger and hostility result directly in increased sympathetic activity leading to arterial constriction and increased blood pressure and heart rate. Emotional factors provide indirect pathways to CVD by influencing behavior and habits, such as smoking, overeating, lack of exercise, and drug usage.

The combination of mental stress and hidden cardiovascular disease can be lethal. A clear case study is that of firefighters. More firefighters die from heart attack than from the direct effects of fire. As can be seen in recent television reports, city fire departments have become increasingly aware of the situation and are implementing new screening procedures for the detection of subclinical cardiovascular disease.

In a military context, seemingly ‘healthy’ military personnel may also be at considerable risk of cardiovascular events, particularly in mission-critical situations with high levels of physical and psychological mental stress. The effects of mental stress on cardiovascular function vary among individuals and cannot readily be predicted, even with the most advanced methodologies. Military personnel can be exposed to extremes of physical and psychological mental stress during active duty, and to considerable mental stressors afterwards. At present there are no practical means to continuously monitor the cardiovascular effects of mental stress in ambulant subjects. Thus, the cardiovascular fitness levels sufficient to tolerate mental stress, and the adverse short- and long-term cardiovascular effects of mental stress cannot be quantified.

Studies have established that CVD occurs, and potentially can be detected, years before normal symptoms would appear. CVD is an insidious disease in that its characteristic symptoms are often manifest only at an advanced stage and under conditions of physiological mental stress. It is now widely known that traditional cardiovascular risk assessment such as blood testing, resting electrocardiogram (ECG) and treadmill mental stress tests fail to identify most individuals at risk of heart attack.

Endothelial function (EF) is becoming accepted as the most sensitive indicator of vascular function. EF has been labeled a “barometer of cardiovascular risk” and is well-recognized as the gateway to cardiovascular disease, by which many adverse factors damage the blood vessel. See Vita J A and Keaney J F Jr. “Endothelial function: a barometer for cardiovascular risk?” Circulation 106(6) (2002) 640-2. EF is impaired in the presence of physiologic and psychological mental stress. Endothelial dysfunction causes impaired vascular reactivity, compounds the adverse effects of inflammatory factors, and underlies a variety of vascular and non-vascular diseases, particularly heart attack and stroke. Conversely, EF improves with positive psychological stimuli. Thus, EF not only predicts risk, but can also parallel changes in response to therapy (pharmacologic and non-pharmacologic) and to alterations in risk factors.

However, traditional techniques for assessment of endothelial function are either invasive or require sophisticated equipment. Such techniques include forearm plethysmography with intra-arterial acetylcholine challenge testing and high-resolution ultrasound imaging of the brachial artery during an arm-cuff occlusion reactive hyperemia test (flow-mediated vasodilatation, FMD). The problems and difficulties associated with the ultrasound imaging such as sensitivity to probe positioning, signal artifacts, poor repeatability, need for skilled technicians, observer dependence, observation bias, and high cost have limited the use of this invaluable test to research laboratories.

Although the standard way of assessing psychological damage due to mental stress has been via counseling and use of questionnaires, various parallel (animal and a few human) studies have attempted to quantify mental stress levels in terms of the assessment of cortisol levels in saliva. See Patacchioli F R, et al. “Actual mental stress, psychopathology and salivary cortisol levels in the irritable bowel syndrome (IBS)” J Endocrinol Invest. 24(3) (2001) 173-7.

With respect to acute detection of mental stress, it has been suggested that mental stress responses can potentially be measured by recording muscle tension by electromyography (EMG), brain waves by electroencephalography (EEG), galvanic skin response (i.e., electro dermal response-EDR), skin temperature, heart rate, blood pressure or eye movements such as pupil dilation, in response to mental stress stimulators. See e.g. U.S. Pat. No. 6,102,846. Certain of these methods including electromyography (EMG), electroencephalography (EEG), and eye movements are not readily adapted to ambulatory assessment. Others of these methods, including heart rate and blood pressure, can change as a consequence of physiologic demand without a component of emotional mental stress.

The relationship between mental stress and peripheral body temperature (e.g. finger temperature) is known. See Shusterman V and Barnea O “Spectral characteristics of skin temperature indicate peripheral mental stress-response” Biofeedback And Self-Regulation 20 (4) (1995) 357-367; Shusterman V and Barnea O “Sympathetic nervous system activity in mental stress and biofeedback relaxation. Monitoring SNS activity with the photoplethysmographic-wave envelope and temperature-variability signals” IEEE Eng Med Biol Mag. 24(2) (2005) 52-7; Shusterman V et al. “Spontaneous skin temperature oscillations in normal human subjects” Am J Physiol. 273(3 Pt 2) (1997) R1173-81. Temperature variability is attributed to changes in blood flow resulting from oscillations in vasomotor smooth muscle tone. It has also been shown that the blood pressure wave response to the mental stress is similar to the oscillatory behavior displayed by the peripheral (cutaneous) temperature under similar mental stress conditions.

However, skin temperature is influenced by physiologic and environmental conditions that confound isolation of mental stress induced components. Thus, use of temperature to monitor mental stress has heretofore required controlled environmental conditions. Examples of methods include, for example, U.S. Pat. No. 4,450,843 disclosing a miniature biofeedback instrument that combines a wrist mounted thermistor for temperature sensing and a piezoelectric transducer for sensing heart beats. In U.S. Pat. No. 6,656,116, a device that can be attached as a wrist strap is provided for perceiving an emotional state including a blood pressure sensor, a skin temperature sensor, and a skin resistivity sensor. In U.S. Pat. Nos. 6,743,182 and 6,520,921, a method is provided for determining the appropriate dosage of a medication to treat Attention Deficit Hyperactivity Disorder (ADHD) by sampling the peripheral skin temperature of affected subjects.

What are needed are methods and apparatus for ambulatory quantitative monitoring of mental stress under real-life conditions with control for non-mental stress induced components of the measured mental stress indicator. Also needed are methods and apparatus to measure and associate mental stress responses with vascular function under real-life conditions such that individuals with hidden susceptibility to pathologic vascular effects of mental stress can be identified.

### BRIEF SUMMARY OF THE INVENTION

The present invention provides methods and apparatus for determining individual responses to induced and actual mental stress in order to identify an individual's vulnerability to the detrimental effects of mental stress on the cardiovascular system. The methods and apparatus of the present invention provide for: 1) ambulatory mental stress monitoring, 2) ambulatory vascular reactivity monitoring, and 3) assessment of the combined effects of impaired vascular function and mental stress. The combined effects of impaired vascular function and mental stress are particularly suited to three population groups: 1) asymptomatic subjects at risk for chronic effects of mental stress by virtue of synergistic effects of mental stress and cardiovascular (CV) disease risk factors including hypercholesterolemia, hypertension, smoking, diabetes mellitus, family history and other CV risk factors (also called susceptible, due to presence of risk factors), 2) subjects at heightened risk to acute effects of mental stress by virtue of synergistic effects of mental stress and existing but asymptomatic CVD such as hidden coronary atherosclerosis (also called vulnerable due to presence of arterial atherosclerosis), and 3) coronary heart disease (CHD) patients at risk of a second heart attack.

One embodiment of the invention provides an ambulatory method for profiling an individual's vulnerability to detrimental effects of mental stress on vascular function. Baseline mental stress and vascular function levels are monitored. During a mental stress challenge, the changes of mental stress and vascular function levels are also monitored and correlated to profile the individual's vulnerability to detrimental effects of mental stress on vascular function.

One embodiment of the invention provides a method for generating an ambulatory record of mental stress induced neurovascular activity by monitoring and recording blood flow differences between a body part having maximal sympathetic nervous system reactivity and a body part that is relatively unaffected by sympathetic stimulus. The blood flow is continuously monitored and recorded during an established test period. The monitoring apparatus is adapted to be worn during outpatient ambulatory activity such that individual responses under various life activities can be determined. However, the method and apparatus are also suitable for monitoring under controlled conditions, including where mental stress is simulated or induced by mental stress inducers known in the art. The pattern of reactivity identifies those individuals having frequent and dangerous mental stress responses. Blood flow differences can be determined by several methods including skin temperature, induced temperature wash-out, Doppler ultrasound, laser Doppler flowmetry, photoplethysmography, and/or changes in magnetic or electromagnetic properties of the tissue.

In one embodiment of the invention, the body part having maximal autonomic reactivity is a fingertip and the body part that is relatively unaffected by autonomic stimulus is a palm of a same hand. In other embodiments, a vascular stimulus is administered during the test period such that the effects of mental stress on vascular reactivity are determined for the individual.

In one embodiment, methods and apparatus are provided for determining psychological or psycho-vascular status in an individual by continuously measuring and recording blood flow on a palm of a hand of the individual while simultaneously and continuously measuring and recording blood flow on a fingertip of the same hand. Differences in blood flow between the palm and the fingertip are determined and mental stress events are correlated with changes in fingertip blood flow corrected for changes in palm blood flow, thereby determining the individual's psychological or psycho-vascular reaction to mental stress. In one embodiment, blood flow on the palm is measured at a plurality of locations on the palm. In other embodiments, measurement of fingertip blood flow is recorded on a plurality of fingers of the hand. Blood flow can be determined by various methods including by measuring inherent skin temperature or, alternatively, by clearance of induced skin temperature. Inherent skin temperature means the unaltered temperature of the skin. This is in contrast to an induced skin temperature measurement which measures perfusion by wash-out of heat induced on the skin. Alternatively, blood flow may be measured by other techniques including tonometry, laser Doppler flowmetry (such as by a laser Doppler perfusion imaging (LDPI) instrument), photoplethysmography and iontophoresis.

In another embodiment of the invention, mental stress is additionally measured by skin neuro signaling activity (cutaneous or subcutaneous sympathetic nerve activity or general neuro stimulation) including by microneurography.

In one embodiment of the invention, the ambient temperature is simultaneously recorded and the fingertip and palm blood flow is compared relative to the ambient temperature. Mental stress may be “normal” situational mental stress to which the individual is exposed or may be induced. For example, mental stress may be induced in a virtual reality simulator or by administration of a chemical mental stress inducer. For situational mental stress, mental stress events may be documented in a log and correlated with the recorded changes in perfusion. The log can be implemented through a “PDA” type electronic entry device in electronic communication with a controller for measuring and recording blood flow on the palm and fingertip.

In one embodiment of the method and apparatus for determining psychological or psycho-vascular status, a reactive hyperemia response induced in the hand is further simultaneously and continuously measured. Reactive hyperemia can be induced by occlusion on a digit proximal to fingertip or by occlusion of a radial artery proximal to the fingertip. Alternatively, reactive hyperemia can be induced by compression of a brachial artery proximal to the fingertip.

In one embodiment, the method for determining psychological or psycho-vascular status further includes simultaneously measuring and recording additional physiologic parameters including pulse rate, blood pressure, galvanic response, sweating, core temperature, and/or skin temperature on the thoracic or truncal (abdominal) part.

In one embodiment of the invention, a method of determining an individual at risk for acute cardiovascular effects of mental stress is providing including measuring ambulatory mental stress responses in the individual; determining a vascular function status in the individual; and determining a relative risk for an acute cardiovascular effect of mental stress considering the ambulatory mental stress response in light of the vascular function status of the individual. Optionally, a cardiovascular risk factor status of the individual can be determined and considered in light of the ambulatory mental stress response. In one preferred embodiment, the ambulatory mental stress response is measured by determining a difference in blood flow between a palm and a fingertip of the same hand of the individual when the individual is exposed to mental stress events. In one preferred embodiment, the vascular function is determined by digital temperature monitoring of a digit subject to an inducer of reactive hyperemia.

In another embodiment of the invention, a method of determining subjects at risk for a second heart attack is provided comprising measuring ambulatory mental stress responses in the individual after release from treatment for a first heart attack, wherein the ambulatory mental stress response is measured by determining a difference in blood flow between a palm and a fingertip of a same hand of the individual when the individual is exposed to mental stress events. Ambient temperatures can be simultaneously recorded and compared with the fingertip and palm perfusion relative to the ambient temperature. Mental stress can be normal situational mental stress of daily life or can be emulated by administration of a chemical mental stress inducer or by subjecting the individual to tests known to induce mental stress or to a virtual reality simulator. A log of mental stress events is correlated with the measured ambulatory mental stress responses.

The method disclosed herein is implemented by an ambulatory device for measuring mental stress having at least one finger mounted blood flow monitor in electrical communication with a control unit, and at least one palm mounted blood flow monitor in electrical communication with the control unit, wherein the control unit is adapted to continuously measure and record data from both finger and palm mounted blood flow monitors. The finger and palm mounted blood flow monitors can be selected from the group consisting of inherent temperature sensors, induced temperature sensors, tonometry sensors, laser Doppler flowmetry sensors, and photoplethysmography sensors. In one embodiment, the apparatus further includes an ambient temperature sensor.

The ambulatory device can further include an occlusion cuff for inducing a reactive hyperemia response in the hand on which the finger and palm sensors are disposed. The occlusion cuff of one embodiment is a finger mounted occlusion cuff disposed proximal or up-stream toward the heart to the finger mounted blood flow sensor. In another embodiment, the occlusion cuff is a wrist mounted occlusion cuff disposed proximal to the finger and palm mounted blood flow sensors. Alternatively, the device may include both finger and wrist mounted occlusion cuffs. The blood flow sensors are in electrical communication with a controller, which may further control the occlusion cuff(s) and may include remote telemetry instrumentation as well as a power supply. In other embodiments, the ambulatory device further includes an additional physiologic parameter monitor selected from one or more of monitors for: pulse rate, blood pressure, galvanic response, sweating, core temperature, and/or skin temperature on the thoracic or truncal (abdominal) part. The controller may further control the additional physiologic parameter monitors.

In one embodiment the ambulatory device is a glove and the elements of the device are disposed on the glove such that the various instrumentalities of the device are applied by putting on the glove. In one embodiment, the glove is composed of a mesh like fabric.

In one embodiment of the invention an ambulatory device for measuring reactive hyperemia is provided including a cuff dimensioned to be worn on a finger, the cuff including a band that can be controllably tightened on the finger thereby restricting an arterial flow distal to the cuff, a release for relieving the restriction of the band thereby restoring arterial flow distal to the cuff, and a sensor for measuring blood flow to a fingertip distal to the cuff. The band can be inflatable or can be a strap that is tightened manually. The sensor can be a temperature sensor. Alternatively the sensor can be fingertip arterial tonometry sensor. The ambulatory device can be disposed in a glove and may further comprise palm blood flow sensors for comparing blood flow to the fingertip and to the palm thereby estimating individual mental stress responses in light of the vascular reactivity measured in the context of reactive hyperemia.

### BRIEF DESCRIPTION OF THE DRAWINGS

FIG. 1 depicts a DTM device for assessment of vascular function under controlled conditions.

FIG. 2a graphically depicts the analyzed parameters from Digital Thermal Monitoring (DTM) data points from a finger on an arm subject to reactive hyperemia (black diamonds). Hypothetical data from a contralateral control finger also shown for purposes of comparison (in grey circles). FIG. 2b schematically depicts various diseases that involve endothelial dysfunction.

FIG. 3A depicts by a reactive hyperemia response by video thermography and by DTM. An example of a fingertip thermal response (DTM) is depicted in FIG. 3B, while a graphic depiction of variations in vascular reactivity is depicted in FIG. 3C. FIG. 3D depicts results from clinical studies showing the correlation with DTM results and the Framingham Risk Score. FIG. 3E shows the formula used to calculate TR as depicted in FIG. 3D.

FIGS. 4a and 4b depict several views of embodiments of a finger cuff and temperature sensor for ambulatory vascular reactivity assessment.

FIG. 5 depicts several views of another embodiment of a finger cuff for inducing reactive hyperemia.

FIG. 6 depicts an embodiment of a hand mounted control and battery pack including an ambulatory pump for controlled inflation of a finger cuff.

FIG. 7 depicts a ventral view of another embodiment of an ambulatory mental stress and vascular reactivity monitor including at least one palm temperature sensor.

FIG. 8 depicts a ventral view of a glove embodiment of an ambulatory mental stress and vascular reactivity monitor including at least one palm temperature sensor.

FIG. 9a depicts a dorsal view of another glove embodiment, while FIG. 9b depicts a ventral view of the wrist region of this embodiment.

FIG. 10 depicts a ventral view of a glove embodiment of an ambulatory mental stress and vascular reactivity monitor including at plurality of finger cuffs and finger temperature monitors as well as a plurality of palm temperature sensors disposed in a temperature sensor array.

FIG. 11 depicts a dorsal view of an embodiment of a finger cap type sensor.

FIG. 12 depicts a dorsal view of a mesh glove embodiment.

FIG. 13 depicts a dorsal view of a mesh glove embodiment having a plurality of finger mounted sensors.

FIG. 14a depicts a ventral view of one embodiment of a sensor wiring harness for fingertip and palm blood flow monitoring, while FIG. 14b depicts a dorsal view of the wrist region of this embodiment depicting a connection between the sensor wiring harness and a wrist band controller.

### DESCRIPTION OF THE INVENTION

Different individuals having the same level of cardiovascular risk factors such as high cholesterol, smoking, diabetes etc may exhibit differences in vascular reactivity and vascular functional responses once exposed to the same level of mental stress. Those with impaired vasoreactivity (less vaso dilative capacity) are more likely at risk of future cardiovascular disease such as heart attack and stroke. However, there are at present no means to readily identify these individuals.

Psychological mental stress and subclinical cardiovascular disease (CVD) interact lethally in certain individuals. Repeated exposure to high levels of physical and, particularly, psychological mental stress, and sustained exposure to low levels of mental stress, both of which are experienced during high mental stress jobs and military service, may impair endothelial function acutely, and cumulatively impair cardiovascular health in the longer term. Mental stress amplifies the interaction between risk factors for atherosclerosis and vascular endothelial dysfunction. Mental stress also impairs mental acuity, awareness, perception, and decision-making ability.

Clinically silent coronary artery disease is well documented in apparently healthy individuals, many of whom are routinely and chronically exposed to episodic psychological mental stress at levels that may promote CVD. Effects of mental stress may manifest suddenly and unexpectedly, as in the case of acute coronary syndromes and sudden cardiovascular death in both civilian and combat-related circumstances. Given inter individual differences in susceptibility, typically subtle and asymptomatic short term CV effects, and the lack of adequate methods to quantify cumulative mental stress exposure, it is impossible to accurately identify those individuals at highest risk of mental stress-dependent CVD, including life-threatening CV events. One embodiment of the present invention provides a method for ambulatory quantitative monitoring of psychological mental stress and vascular function, to identify individuals with hidden susceptibility to pathologic vascular effects of mental stress in order to prevent death, disability, and high costs associated with cardiovascular and mental vulnerability to mental stress.

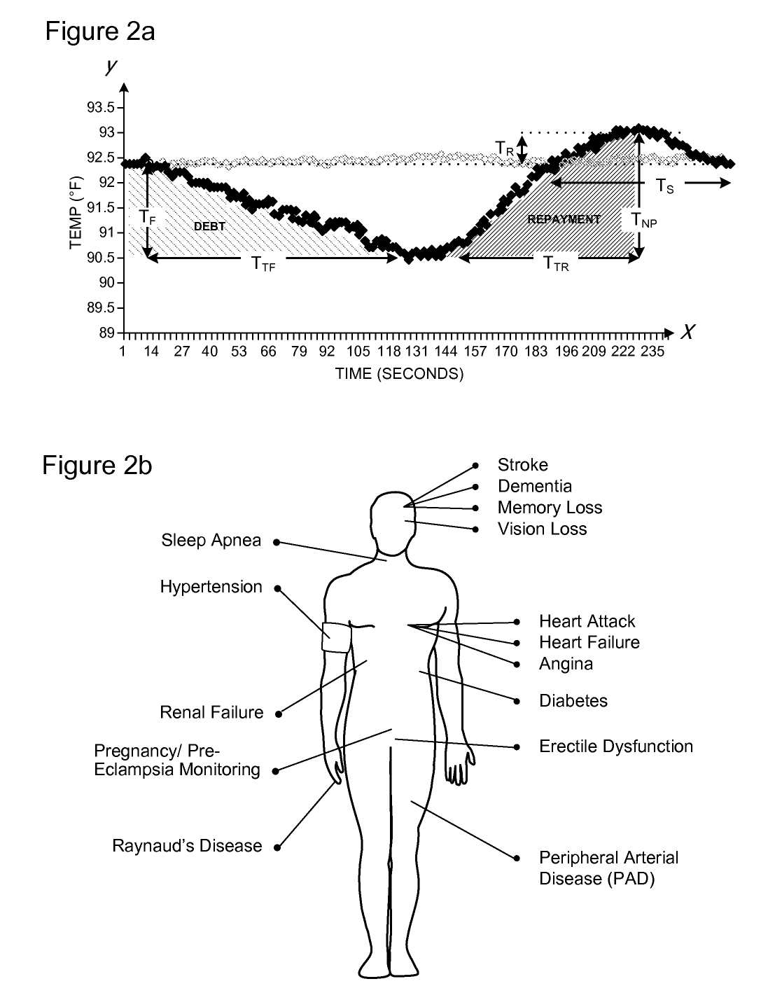

Vascular Responses Mediated by the Endothelial System: All of the blood vessels in the body are lined by a single layer of cells known as the vascular endothelium. Endothelial dysfunction causes impaired vascular reactivity, compounds the adverse effects of inflammatory factors, and underlies a variety of vascular and non-vascular diseases, particularly heart attack and stroke. Certain of the diseases associated with endothelial dysfunction are depicted graphically in FIG. 2B. Endothelial dysfunction is correlated with several risk factors, including familial hypercholesterolemia, smoking, diabetes mellitus, and hyperhomocysteinemia. Repeated exposure to high levels of physical and, particularly, psychological mental stress, and sustained exposure to low levels of mental stress, both of which are experienced during active duty and high mental stress jobs, may impair endothelial function acutely, and cumulatively impair cardiovascular health in the longer term.

Endothelial function can be evaluated by various different approaches, including: measurement of structural characteristics of the vascular wall, e.g. intima media thickness, compliance, distensibility, and remodeling indexes; measurement of soluble endothelial markers including von Willebrandt factor, plasminogen activator, inhibitor complex thrombomodulin adhesion molecules, and nitric oxides; and measurement of endothelium-dependent regulation of vascular tone. See Kelm M. “Flow-mediated dilatation in human circulation: diagnostic and therapeutic aspects” Am J Physiol Heart Circ Physiol 282 (2002) H1-H5.

Endothelium-dependent vasodilation as a measure of endothelial function can be determined by invasive vasomotor techniques including quantitative coronary angiography and strain gauge plethysmography of the forearm with intra-arterial acetylcholine challenge. Due to the invasive nature of these methods, brachial artery flow-mediated dilation (FMD) measurement by high-resolution ultrasonography has been alternatively accepted as a research tool, albeit highly technical, for the examination of endothelial function. See Sorensen K E, et al. “Non-invasive measurement of human endothelium dependent arterial responses: accuracy and reproducibility” Br Heart J 74 (1995) 247-253. Brachial artery flow-mediated dilation (FMD) measurement by high-resolution ultrasonography utilizes the phenomena of reactive hyperemia. Reactive hyperemia is defined as hyperemia, or an increase in the quantity of blood flow to a body part, resulting from the restoration of its temporarily blocked blood flow. When blood flow is temporarily blocked, tissue downstream to the blockage becomes ischemic. Ischemia refers to a shortage of blood supply, and thus oxygen, to a tissue. When flow is restored, the endothelium lining the previously ischemic vasculature is subject to a large, transient shear mental stress. In partial response to the shear mental stress, the endothelium normally mediates a vasodilatory response known as flow-mediated dilatation (FMD). The vasodilatory response to shear mental stress is mediated by several vasodilators released by the endothelium, including nitric oxide (NO), prostaglandins (PGI₂) and endothelium-derived hyperpolarizing factor (EDHF), among others. A small FMD response is interpreted as indicating endothelial dysfunction and an associated increased risk of vascular disease or cardiac events. See Pyke K E and Tschakovsky M E “The relationship between shear mental stress and flow-mediated dilatation: implications for the assessment of endothelial function” J Physiol 568(2) (2005) 357-9.

Induction of reactive hyperemia is well-established in clinical research as a means to evaluate vascular health and in particular endothelial function. Typically, a reactive hyperemia procedure is implemented by occluding arterial blood flow briefly (2-5 minutes, depending on the specific protocol) in the arm, by supra-systolic inflation of a standard sphygmomanometer cuff, then releasing it rapidly to stimulate an increase in blood flow to the arm and hand. Reactive hyperemia has been classically measured by high-resolution ultrasound imaging of the brachial artery during and after arm-cuff occlusion. However, the technical difficulties of ultrasound imaging have limited the use of this test to research laboratories. This method is clearly unsuitable to widespread adoption of reactive hyperemia as a test of vascular function. The method is simply inapplicable to evaluation of endothelial function in the context of real life mental stress inducers.

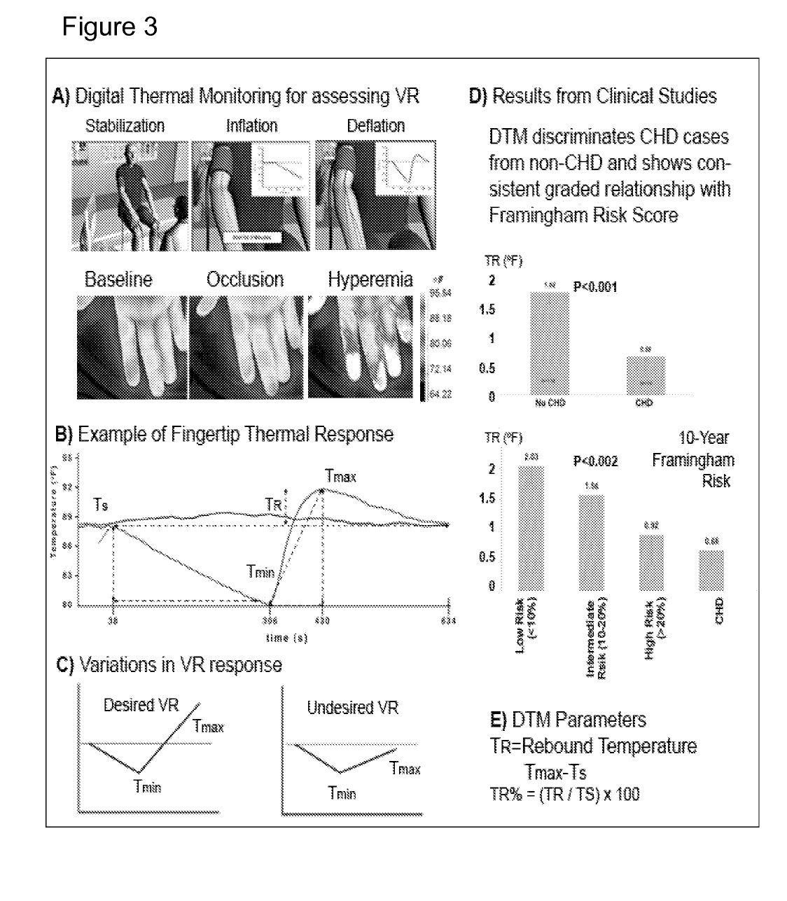

Digital Thermal Monitoring: Certain of the present inventors have previously developed novel methods and apparatus to determine the vascular reactivity based on a measured response of the vasculature to reactive hyperemia utilizing continuous skin monitoring of inherent temperature on a digit distal (downstream) to an occluded arterial flow. By inherent temperature it is meant the unmodified temperature of the skin as opposed to measurement of the dissipation of induced temperature. This principal and technique has been termed Digital Thermal Monitoring (DTM). See WO 05/18516, the disclosure of which is incorporated herein by reference. DTM is typically implemented by measuring temperature changes at the fingertips during reactive hyperemia induced by transient arm-cuff occlusion and subsequent release. A normal reactive hyperemia response, i.e. increased blood flow after occlusion, is manifest by increased skin temperature over the baseline temperature established prior to occlusion. FIG. 3A depicts the steps of a DTM assessment and shows, in the bottom panel, a thermographic record of the cooling of the hand and fingers as a consequence of arm-cuff occlusion as well as the rebound temperature after release of the cuff that exceeds that of baseline in an individual with a good vascular response. Since endothelial function is a systemic property, a localized measurement in a readily accessible location of the human body (such as the digits) can provide an accurate assessment of vascular health in physiologically critical locations such as the coronary arteries. DTM is thus being developed as a new surrogate for endothelial function monitoring that is non-invasive, operator-independent (observer-independent) and is sufficiently straightforward to be readily implemented across the population to assess individual vascular function. Preliminary studies, as described below, have shown that DTM can discriminate individuals with established CHD or high risk of future CHD (as measured by Framingham Risk Score) from normal and low-risk individuals.

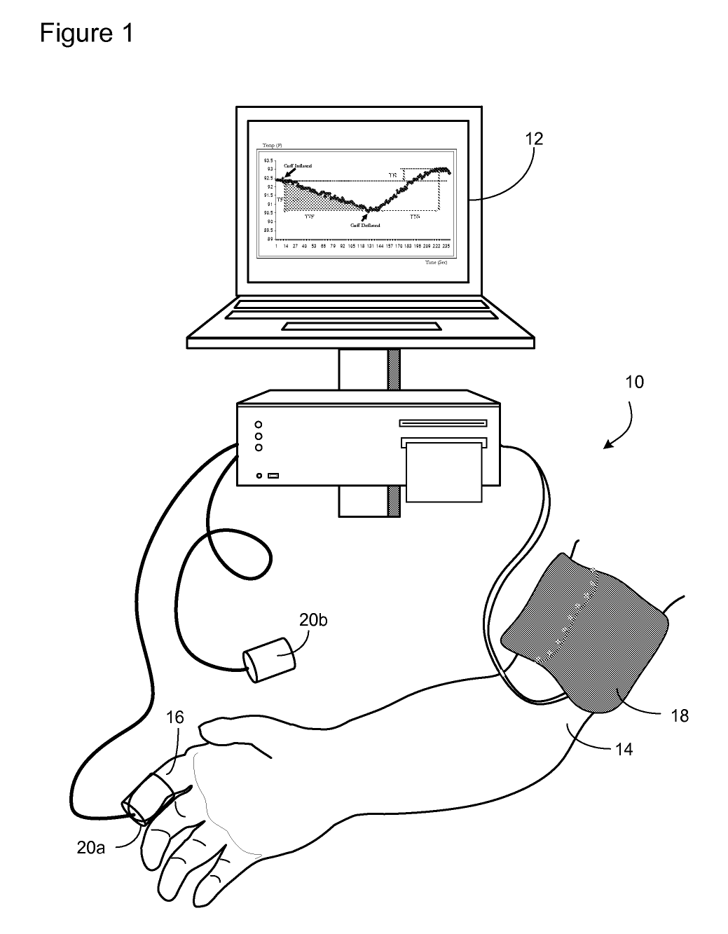

A pilot study was performed with the aim of evaluating the potential clinical utility in cardiovascular risk stratification of DTM. DTM was performed using a VENDYS ® DTM system 10 generally as depicted in FIG. 1. Reactive hyperemia is induced through transient inflation of cuff 18 placed on arm 14. Skin temperature is detected by temperature sensor 20a placed on a finger 16. Temperature sensor 20b is placed on a respective finger on the contralateral hand as an internal control. In the pilot study described herein, the temperature sensor employed was a thermocouple. However, other temperature sensors might be alternatively employed in the implementation of DTM, including Resistance Temperature Detectors (RTM), thermisters, thermopiles or integrated circuit (IC) detectors.

DTM assessment is conducted generally as follows. In a standard controlled setting, the subject is seated and the cuff 18 is placed on one arm 14. Temperature probe 20a is placed on the index finger of the cuffed arm and temperature probe 20b is placed on the index finger of the contralateral arm. Baseline temperature data is continuously recorded for an equilibration period, for example three minutes. The cuff 18 is inflated rapidly to 200 mm Hg or 50 mm Hg above systolic blood pressure and the pressure is retained at this level for 2 to 3 minutes. During this period skin temperature falls on the fingertip of the occluded arm. After 2 to 3 minutes, the cuff is rapidly deflated and the skin temperature rapidly rises as blood returns to hand and fingers. Temperature is recorded for another 3 minutes after the cuff is deflated and the data from both fingers is captured and displayed by a computer 12. The following primary parameters are calculated as depicted in part in FIG. 2a.

Measures reflecting the ischemic stimulus/thermal debt:
T_{S} Starting fingertip temperature
Tₘᵢₙ (Nadir (N)) Lowest temperature observed after cuff inflation
TF Temperature Fall, T_{S}-Tₘᵢₙ
TTF Time from cuff release to TF (tₘᵢₙ₋tᵢ)
tᵢ Time when the initial temperature was recorded
tₘᵢₙ Time taken to attain Tₘᵢₙ
tₘₐₓ Time to attain maximum temperature
t_{f} Time to attain the equilibrium temperature (final temperature).

Parameters reflecting thermal recovery/vascular reactivity:
Tₘₐₓ Highest temperature observed after cuff deflation
TR Tₘₐₓ−Tₛ (temperature recovery/rebound)
NP Nadir-to-Peak, Tₘₐₓ-Tₘᵢₙ
TTR Time from cuff release to TR, (tₘₐₓ₋tₘᵢₙ)
Slope Slope of temperature recovery=NP/(TTR)
AUC Area under the temperature-time curve

TR and NP indicate the vasodilatory capacity of the vascular bed (small arteries and micro-vessels) and subsequent hyperemia induced brachial artery dilation. TR and NP indicate the vasodilatory capacity of the vascular bed (small arteries and micro-vessels) and subsequent hyperemia induced brachial artery dilation. TR specifically denotes the ability of the arterial bed to compensate for the duration of the ischemia and to create an overflow (hyperemia) above the baseline level. Given a good vasodilatory response and constant room temperature one would expect a positive TR. The higher the TR, the higher the vasodilatory response of the arterial bed. TR close to zero indicates a lack of strong vasodilatory response and negative TR is likely to represent a vasoconstrictive response. NP and TR largely overlap and both show similar information with TR being a more sensitive marker of overflow (hyperemia response) and NP showing additional factors that affect TF (such as neuroregulatory effect and basal metabolic rate). Factors as TTF, TTR and area under the curve are expected to provide additional insights into the response to the ischemia challenge test.

In preliminary studies, several parameters including TF, TR, NP, TTR, TTF were measured. These parameters were correlated against two standard methods of estimating blood flow changes in the forearm: flow-mediated dilatation of the brachial artery, and strain-gauge plethysmography, both during reactive hyperemia in apparently healthy volunteers. In one study, DTM results were compared against Framingham Risk Estimation (FRE) in a community setting. 133 subjects, responding to a local newspaper advertisement, gave informed consent to participate in this study. Subjects agreed to disclose limited medical information regarding any history of cardiovascular disease and cardiovascular risk factors, to a finger stick blood draw for non-fasting lipid profile measurement, and to undergo DTM on up to 3 occasions. Subjects fasted overnight and refrained from smoking, alcohol or caffeine ingestion and use of any vasoactive medications on the day of the testing in both protocols. Subjects remained seated, with the forearms supported at knee level. VENDYS™ DTM probes were affixed to the index finger of each hand as previously described.

In these preliminary studies, DTM appeared to complement FRE in distinguishing between cohorts with and without self-reported CVD. FIGS. 3A-E depict examples and results of DTM assessments of endothelial function. FIG. 3A depicts Digital Thermal Measurement (DTM) response during and after brachial artery occlusion, the thermographs indicate temperature change during the procedure. FIG. 3B depicts fingertip temperature variation recorded with VENDYS system during VR studies for occluded and not occluded hand. FIG. 3C graphically depicts variations in thermal vascular reactivity response observed in volunteers. FIGS. 3D & E summarize results from clinical studies conducted to assess the predictive value of DTM in CVD. DTM was shown not only to correlate the FRE but offered advantages over prior techniques including: 1) low cost, 2) high sensitivity (with good specificity), 3) ease of use as a self-contained unit, and 4) reproducibility of diagnostics across a subject sample.

One embodiment of the present invention now provides novel methods and apparatus that utilize the principal of reactive hyperemia for ambulatory quantitative monitoring of psychological mental stress and vascular function to identify individuals with hidden susceptibility to pathologic vascular effects of mental stress. Thus, assessment of individual vascular reaction to real-life mental stress inducers is available for the first time.

Mental Stress Effects and the Vascular System: Psychological factors such as mental stress, anxiety, and depression show significant correlations with measurable physiological parameters (such as blood glucose levels, peripheral body temperature, and risk factors for cardiovascular disease). Mental stress also results in the secretion of cortisol which affects the blood sugar levels (abnormal levels can lead to diabetes), immune responses, and can also elicit inflammatory responses.

The relation between mental stress and temperature can be understood as follows. The cardiovascular mechanisms that regulate skin temperature in the hands and feet are closely linked with the activity of the sympathetic division of the autonomic nervous system. Upon activation of this system, the smooth muscles surrounding the blood vessels under the skin surface vasoconstrict, resulting in decreased blood flow to the capillaries and capillary beds (body tissue) near the skin surface. Under mental stress, blood flow through the peripheral capillaries and tissues near the skin surface decreases, and the temperature of the skin decreases. To achieve homeostasis (i.e. return to an unstressed mental state), there is an increase in skin temperature as a result of vasodilatation, or relaxation of the smooth muscles surrounding the peripheral blood vessels. Vasodilatation is usually accompanied by a relaxation of sympathetic activity. There is generally an interval of several seconds between vasodilatation and skin temperature increase, because a certain time period must elapse while the increased amount of blood flows into the capillaries and tissues.

Accordingly, vascular reactivity can be affected by mental stress. In an embodiment, the degree with which mental stress affects vascular reactivity can vary according to an individual's resistance to mental stress. In an embodiment, a vascular reactivity difference in mental stress response can be measured between mental stress resistant and vulnerable individuals. In an embodiment, a baseline TR can be measured prior to a mental stress challenge and compared to a TR measured during or soon after a mental stress challenge. Individuals who are resistant to mental stress can maintain a TR at the same level or greater than their baseline measurement. Individuals who are vulnerable to mental stress can have difficulty maintaining baseline TR levels during or after incidences of mental stress. Accordingly, individuals who have cardiovascular vulnerability to mental stress can be profiled as having reduced TR levels during or after a mental stress response.

Individualized Ambulatory Assessment of Mental stress Reactions: Psychological mental stress and subclinical cardiovascular disease (CVD) interact lethally in certain individuals. In animals, including humans, acute psychological mental stress induces a defense reaction mediated by increased sympathetic nerve activity which in turn elicits the hemodynamic responses of increased heart rate, cardiac output, mean arterial pressure, which together with decreased renal blood flow, result in increased blood flow to the skeletal muscle of the limbs. However, in susceptible individuals, these hemodynamic responses are exaggerated and may trigger acute adverse cardiac events. Chronic effects are also implicated as mental stress amplifies the interaction between risk factors for atherosclerosis and vascular endothelial dysfunction. Given inter-individual differences in susceptibility, typically subtle and asymptomatic short term CV effects, and the lack of adequate methods to quantify cumulative mental stress exposure, it has been heretofore impossible to accurately identify those individuals at highest risk of mental stress-dependent CVD, including life-threatening CV events. The present inventors have developed methods and apparatus able to provide individualized assessment of mental stress reactions that is able to isolate the mental stress response from the confounding variables of general physical and environmental condition.

One embodiment of the present invention relies on continuous measurement of blood flow at anatomic locations with maximum sympathetic nervous system effects, such as the fingertip, relative to blood flow at anatomic locations with minimum sympathetic nervous system effects in order to provide a catalogue of neurovascular responses in a given individual. By continuous measurement, it is meant a series of repeated closely spaced measurements over a test period. The test period might be during the duration of a discrete administered mental stress test or series of tests or might be over a longer duration such as a period of hours or days.

In one embodiment, blood flow is measured by skin temperature and a combination of finger mounted thermal energy sensor and palm mounted temperature sensor is used to distinguish a neurovascular response (autonomic nervous system—ANS—response) that is maximally detected by monitoring fingertip (cutaneous) blood flow and its surrogate (e.g. fingertip temperature) from areas where the ANS effect is not maximum such as core temperature, skin temperature on the thoracic or truncal (abdominal) part, or else where the blood flow, unlike fingertip, is not maximally ANS affected. The closest location to the fingertip is on the palm. Therefore, monitoring the differential (delta) temperature of palm versus fingertip can provide an indicator of ANS activity and thus serve as a surrogate marker of mental stress. One embodiment of this differential monitoring can be achieved by placing a wireless (or wired) thermosensor patch on the skin of the body elsewhere (e.g. chest, abdomen, etc) which would enable simultaneous differential thermal monitoring.

In order for mental stress responses and vascular function to be assessed in the context of real life situations, including mental stress situations, miniaturized wearable devices can be adapted to not interfere with real-life activities but able to isolate and identify neurovascular mental stress responses. By providing a continuous record of neurovascular mental stress responses, the present invention is able to identify those individuals for whom intervention is medically indicated.

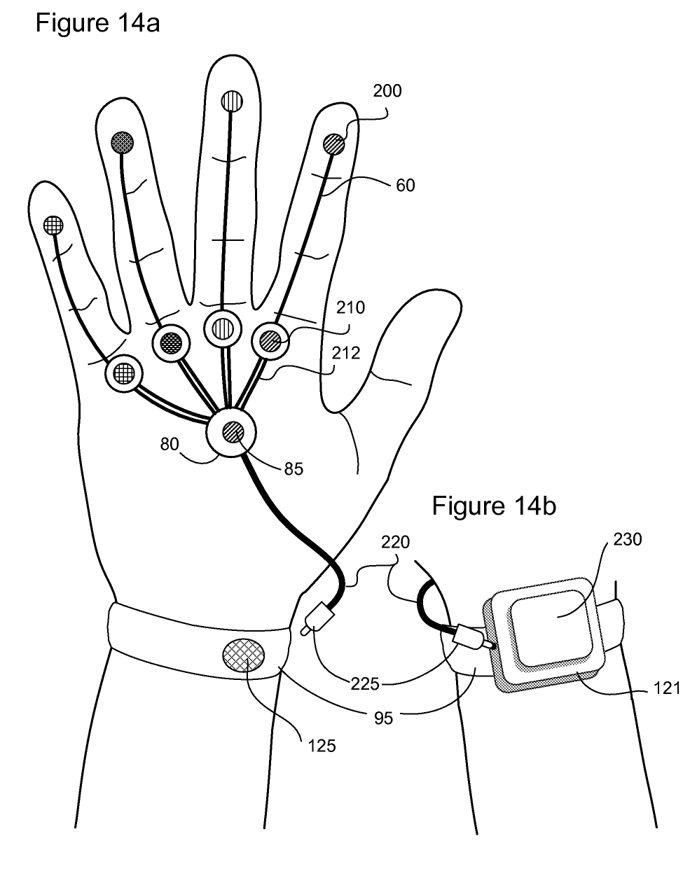

In one embodiment of the invention, a chosen location with maximum sympathetic nervous system effects is the fingertip. In one embodiment, as depicted in FIG. 14a, blood flow is measured by a combination of finger mounted blood flow sensors 200 and palm mounted blood flow sensors 210 and 85. A single finger sensor and palm sensor or a plurality of finger and palm sensors may be variously employed. FIG. 14a depicts an embodiment in which the blood flow on each of the fingertips is monitored as well as a plurality of palm locations. The fingertip sensors and palm sensors are in electrical connection with controller 230. The sensors may be optionally supplied as a sensor and wiring harness wherein each finger sensor is identified by a different color and/or number for attachment to the fingers in accordance with coded instructions. In one embodiment, the sensors are supplied with adhesive disks or backings 80 and may include protective covers for distribution wherein the covers are peeled off for attachment of the sensors. As depicted in FIG. 14a and FIG. 14b (dorsal aspect), the wiring harness is connected electrically to the controller 121, through wiring bundle 220. The controller 121 can be secured to the wrist via band 95, which may optionally include a pulse sensor 125. In operation, the device is virtually unapparent to the casual observer. However, if desired, after application and set-up of the sensor and wiring harness, a light weight mesh glove can be optionally placed over the hand to provide added protection to the assembly. In one embodiment, the glove is of a thin transparent light-weight fabric such as is used in “nylon” stockings. The controller may include some or all of the controlling and recording electronics and may be provided with an input console 230. Alternatively, the controller may be programmed and its data downloaded by communication with a further device such as a PDA. Communication with the PDA may be through a cable hot sync or wirelessly such as for example using existing Bluetooth technology.

In one embodiment, blood flow is measured by skin temperature and the sensors 200, 210 and 85 are temperature sensors, for example thermister or thermocouple temperature sensors. In one embodiment, the temperature sensors are designed as thin, flexible, multijunction thermocouple arrays having electrical sensor junctions spaced at intervals over the length of the finger and onto the palm such that a temperature gradient can be recorded along the length of the array after calibration for any thermal conductivity effect (k effect) along the multicouple wires in accordance with procedures known in the art. See e.g. M. B. Ducharme and J. Frim “A multicouple probe for temperature gradient measurements in biological materials” J Appl Physiol 65 (1988) 2337-2342.

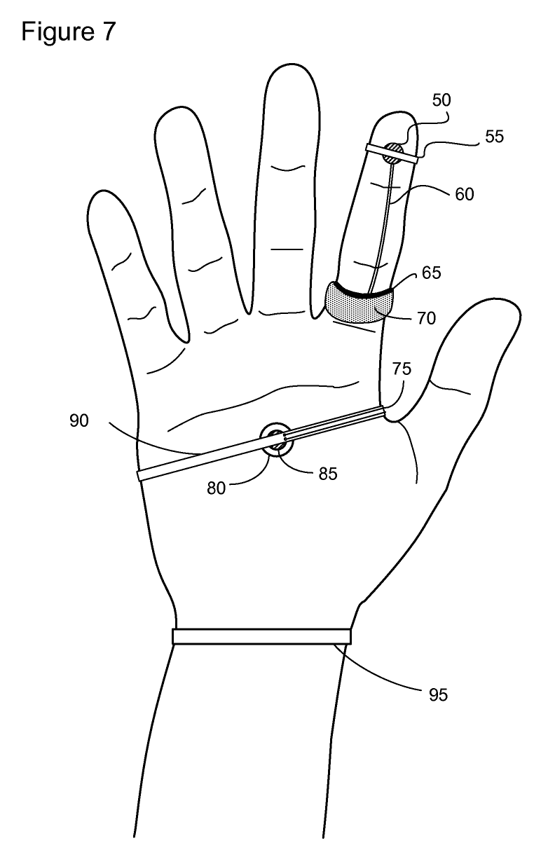

In the embodiment depicted in FIG. 7 a combination of a finger mounted thermal energy sensor 50 and a palm mounted temperature sensor 85 is provided. The thermal energy sensor 50 is held in place with a fingerstrap 55. Optionally, in one embodiment, the thermal energy sensor 50 may include an adhesive patch to adhere the sensor. The thermal energy sensor 50 is in electrical connection via wires 60 to the ring 70. Wires 70 can also be optionally affixed to the finger by an adhesive strip to protect and secure the wire. Alternatively, the temperature sensor is a wireless device. One example of a suitable miniature wireless temperature sensor is disclosed in Wigley et al., U.S. Pat. No. 6,847,913, which discloses a wearable “Band-Aid” sized temperature device for assessing vasospasticity manifest as Raynaud's Syndrome on the fingers.

In further embodiments, the finger and palm monitor combination can further include a vascular occlusive cuff, such as a cuff mounted on the base of the finger or on the wrist to induce reactive hyperemia. In one embodiment, the ring 70 is provided with an occluding strap or inflatable cuff 65 for implementing a reactive hyperemia test in conjunction with DTM. Using this combination, neurovascular responses can be determined and compared with hyperemia vascular reactivity response using thermal monitoring. The present invention also provides a solution for obtaining accurate measurements of mental stress that discriminates between neurovascular responses (autonomic response) from hyperemia vascular reactivity responses. Furthermore, the combination of mental stress monitoring and vascular responsiveness in the context of reactive hyperemia provides for a determination of the effects of mental stress on vascular responsiveness, a particularly relevant physiologic correlation in individuals at risk for mental stress related acute and chronic cardiovascular disease.

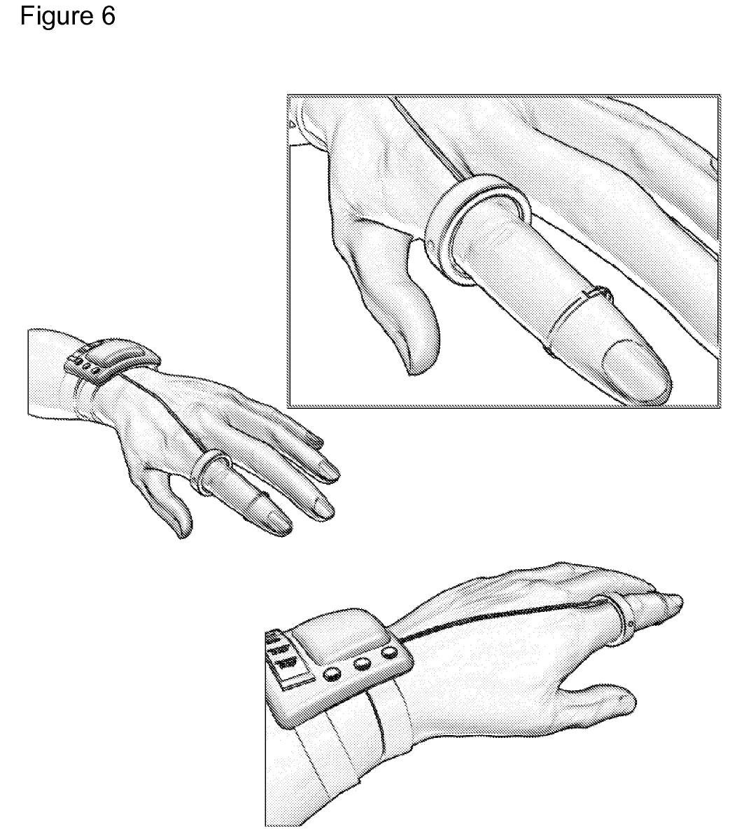

The palm temperature sensor 85 may be held in place with strap 90. Alternatively or additionally, an adhesive patch 80 may be employed to adhere the sensor to the skin of the palm. Wires 75 communicate temperature data from temperature sensor 85. Temperature data from both sensors 50 and 85 is collected simultaneously and conveyed to a control unit (not shown) conveniently mounted in electrical communication with the sensors. For example, the control unit can be mounted on the back of the hand or on the wrist and secured by further strap 95. One such control unit is depicted in FIG. 6.

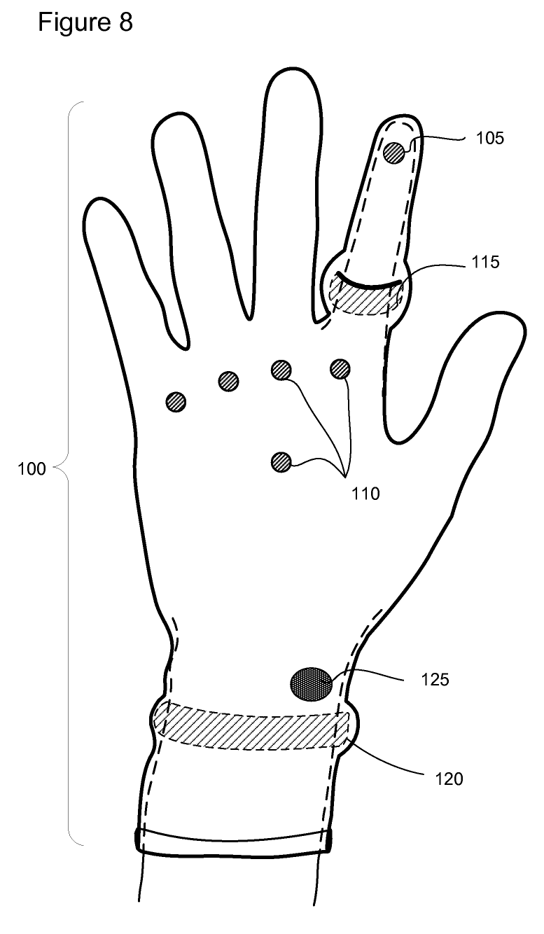

In another embodiment of the invention, depicted in FIG. 8, a glove 100 is provided that includes at least one finger and at least one palm temperature sensor. For vascular reactivity monitoring, the glove includes either or both of a finger occlusion cuff 115 and a wrist occlusion cuff 120, as well as a manual or electric pump (not shown) for controlled inflation of the finger or wrist cuff. Vascular reactivity can be monitored by DTM, or alternatively, by other methods of measuring vascular reactivity including fingertip arterial tonometry (for example using a device available from Itamar Medical) or heat-flux metering (for example using a perfusion device by Hemedex Inc).

Optionally, the glove includes a pulse sensor 125, mounted over the radial artery. One example of a suitable pulse sensor is an oscillometric pressure sensor. The glove also optionally includes one or more of an ambient temperature sensor and a galvanic skin response (i.e., electro dermal response-EDR) monitor. Each of the functional elements of the glove is in electrical communication with a controller mounted on the glove, such as on the back of the hand of the glove or on the top of the wrist. The glove can also include cuff inflation and deflation (tools) mechanisms (built in) to occlude the blood flow (at wrist or elsewhere) for reactive hyperemia and vascular reactivity testing.

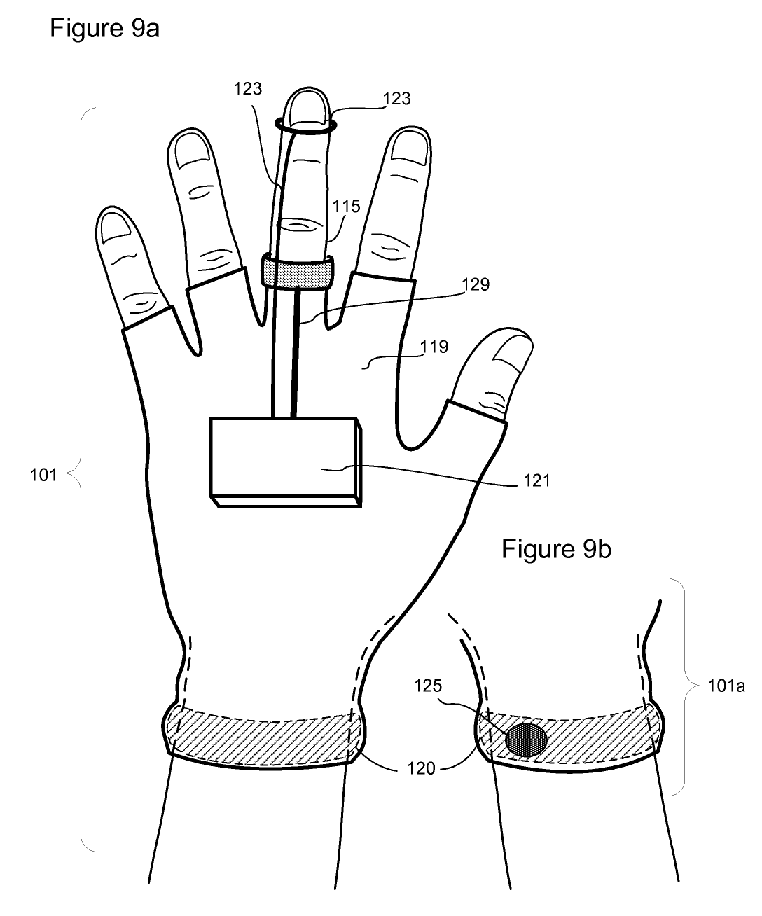

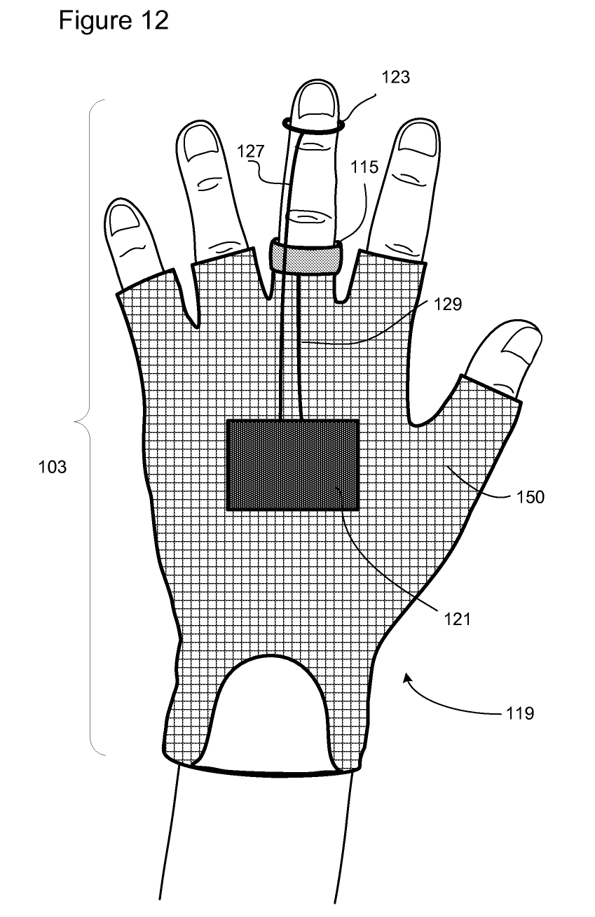

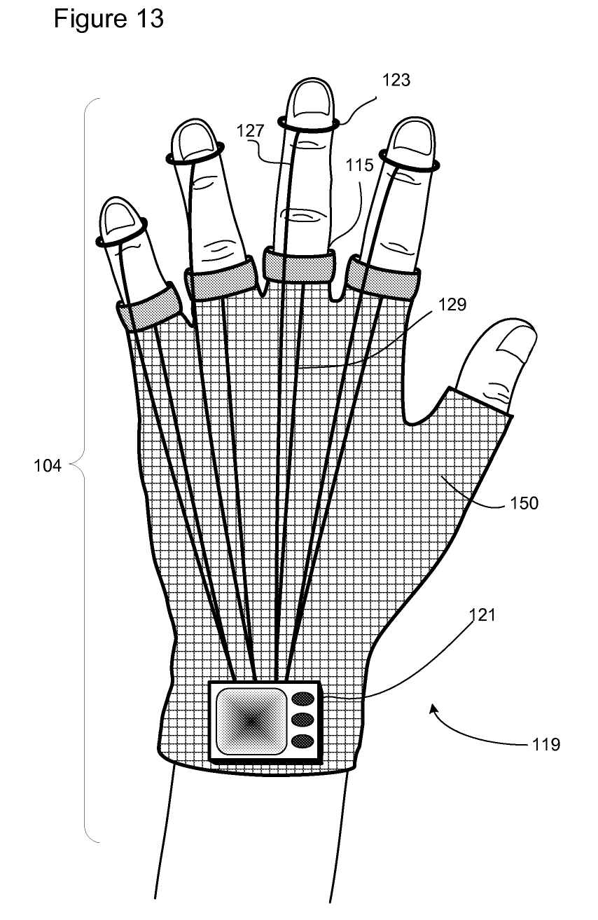

In one embodiment of a glove system, such as that depicted in FIGS. 12 and 13, the fabric 150 of the glove 119 is adapted to allow for heat exchange with the environment such that changes in skin temperature due to cuff occlusion can be detected, assuming the ambient temperature is cooler than core temperature. As depicted in FIGS. 12 and 13, the fabric is an elastic mesh fabric. In one embodiment such as that depicted in FIGS. 9, 10, 12 and 13, the glove design leaves the fingertips out (like a biker glove or a gym glove). As depicted in FIGS. 9, 12 and 13, the controller or processor 121 may be mounted on the glove 119 such as on the dorsal side of the glove as depicted. Controller or processor 121 is connected to the finger occlusion cuff(s) 115 by wire(s) 129 or can alternatively be connected wirelessly such as by RF. Similarly, finger temperature sensor 123 is in electrical connection with controller 121 by wire 127 or is, alternatively connected wirelessly such as by RF connection. As depicted in FIG. 9b, the ventral side 101a of the glove may include a pulse sensor 125 mounted over the radial artery, and if desired the pulse sensor 125 may be located in conjunction with a wrist occlusion cuff 120. As depicted in FIG. 13, a plurality of finger monitors and finger occlusion cuffs may be implemented in the glove system 104.

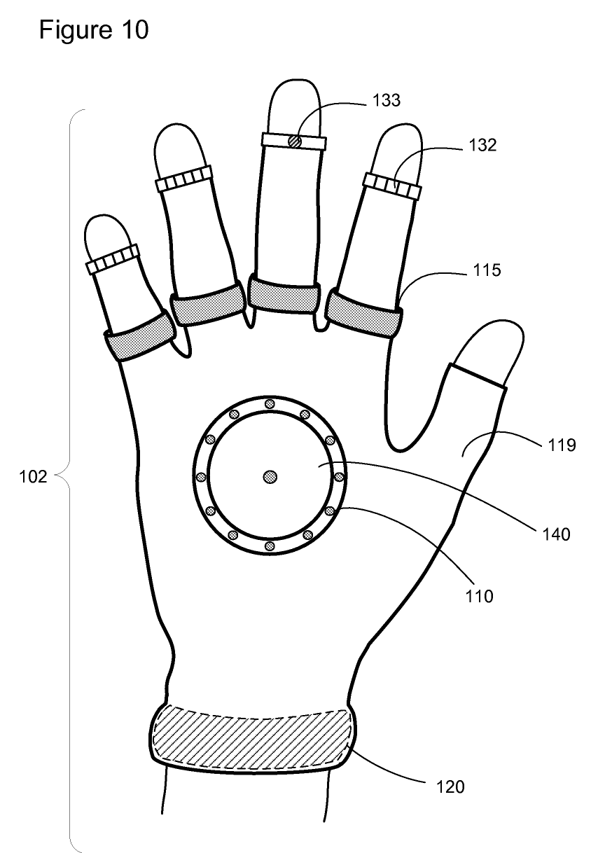

In one embodiment, an example of which is depicted in FIG. 10, the palm perfusion monitor comprises a temperature sensor array 140, including a plurality of temperature sensors 110. In one embodiment depicted graphically in FIG. 10, a glove embodiment is provided in which the ends of the fingers are exposed with finger tip perfusion sensors disposed in the gloves at a location for detecting temperature over the pulp of the fingertips. In one embodiment, the perfusion sensors are temperature sensors such as thermopile sensors 132 or thermocouple sensors 133. Other types of temperature sensors such as RTD sensors may be alternatively employed.

In one embodiment of the invention, a mental stress challenge test is employed to identify a hyperactive sympathetic nervous system and thus to identify those individuals who are prone to develop sustained hypertension. Responses are monitored for an increase in vasoconstriction by looking at increased temperature rather than increased blood pressure. The sympathetic nervous response is assessed for response to mental stressful tests, i.e. challenging mathematical problems or mental stressful movies/pictures. Temperature of the fingertip and palm are continuously measured. A determination of the relative hyperactivity of the sympathetic nervous system is based on the behavior of palm and fingertip temperature before, during and after the mental stress challenge test. This test can be combined with other markers of mental stress, e.g. temperature response along with heart rate or respiratory rate or blood pressure or skin galvanic response to further evaluate the body's reactivity to mental stress. Thus in one embodiment of the present invention, mental stress is assessed during real-life activities by utilizing combined finger and palm temperature monitoring.

In one embodiment of the invention, a miniaturized device is employed to continuously measure and provide for recording of skin and ambient temperature. Because ambient temperature is also recorded, the skin temperature is provided with a contemporaneous reference. In one embodiment a method is provided to determine an individual's reaction to induced mental stress. A finger mounted miniaturized temperature probe is affixed to a finger and can be additionally be mounted to the palm of the same hand. Temperature recording begins, including baseline temperature recordings. Mental stress monitoring by continuous skin temperature recording is combined with real and induced mental stress situation to provide individual assessments of mental stress responses. Under mental stress, blood flow through the peripheral capillaries and tissues near the skin surface decreases, and the temperature of the skin decreases. To achieve homeostasis (i.e. return to unmental stressed state), there is an increase in skin temperature as a result of vasodilatation, or relaxation of the smooth muscles surrounding the peripheral blood vessels. Vasodilatation is usually accompanied by a relaxation of sympathetic activity. A vasoconstrictive response induced by a sufficiently mental stressful situation is normal and may be desirable. However, a vasoconstrictive response to a condition that should not evoke a profound mental stress response is undesirable. Furthermore, the intensity and duration of the response may indicate an inappropriate mental stress response. In one embodiment of the present invention, ambulatory mental stress monitoring by continuous skin temperature measurement is employed to identify dangerous mental stress responses. In another embodiment, ambulatory mental stress monitoring by continuous skin temperature measurement is employed as an objective biofeedback reporter to teach control of mental stress responses. In another embodiment, ambulatory mental stress monitoring by continuous skin temperature measurement is employed as an objective reporter of the success of therapies for mental stress control including by pharmacologic interventions.

In another embodiment, vascular reactivity is assessed during real-life activities by utilizing the finger based cuff occlusion of the present invention to implement reactive hyperemia and measure vascular reactivity by DTM and/or fingertip arterial tonometry (for example using a device available from Itamar Medical). The device is worn in ordinary conditions, normally considered to be non-mental stressful, to establish a “normal” individual vascular reactivity profile. The individual is then subject to various mental stressful conditions to determine that individual's vascular reactivity under mental stress.

DTM assessment can be conducted generally similar to the description related to arm cuffs herein. In a standard controlled setting, the subject is seated and a suitable cuff can be placed on at least one finger. A temperature probe can be placed on the tip of the index finger of the cuffed finger and a temperature probe can be placed on the index finger of the contralateral arm. Baseline temperature data can be continuously recorded for an equilibration period, for example three minutes. The finger cuff can be inflated rapidly to 200 mm Hg or 50 mm Hg above systolic blood pressure and the pressure is retained at this level for 2 to 5 minutes. During this period skin temperature falls on the fingertip of the occluded finger. After 2 to 5 minutes, the cuff is rapidly deflated and the skin temperature rapidly rises as blood returns to hand and fingers. Temperature is recorded for another 3 minutes after the cuff is deflated and the data from both fingers is captured and displayed by a computer. The DTM primary parameters using finger cuffs are calculated along the same curve using arm cuffs as depicted in part in FIG. 2a.

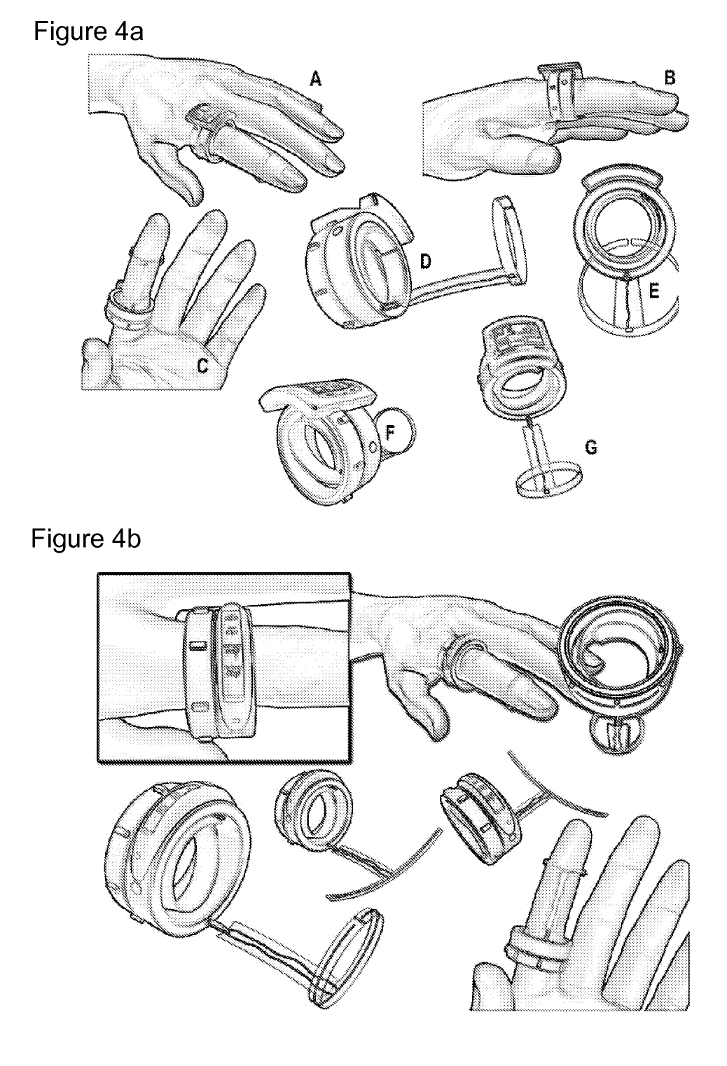

One embodiment of a miniature DTM device (MDTMD) is shown in FIG. 4A. Another embodiment is shown in FIG. 4B. The MDTMD is placed on a finger such as the index finger, and is dimensioned such that the device does not interfere with normal functioning. The embodiment of the device depicted in FIGS. 4A and B consists of three sub-units: (a) an occluding band placed close to the base of the finger. The band consists of two rings, one stationary that has a display unit mounted on it, and the other which can be twisted so as to deploy the inflation. Both are connected with a thick band that enables the tightening mechanism and ensures a snug and comfortable cushioning. Sub-unit (b) is a temperature sensing band placed closer to the finger tips, and sub-unit (c) is a data acquisition and transmission system (DATS), mounted on the occluding band. This system also contains a display unit that shows the pressure and temperature reading along with a sensor. In one embodiment, a remote telemedic computer system receives, analyzes and presents the data to medical staff almost instantaneously. In other embodiments, optional additional measuring devices may include an oximeter, which records the instantaneous heart rate of an individual, and a plethysmographic device to read the blood pressure.

Device functionality is briefly described below, elaborating on the physical operating principles. Upon activation, the occluding band first compresses the artery in the finger, causing ischemia (i.e. interruption in the flow of blood to the finger tips). After a pre-set or programmable occlusion time, the finger tips—having been deprived of normal blood circulation—attain a reduced surface temperature closer to ambient. Following this period of constriction, the occluding band can be manually loosened by pressing a button on the occluding band, thereby immediately restoring blood flow. The subsequent time-variations of the finger-tip temperature are measured by the sensor.

Referring again to FIG. 4A depicting an embodiment of a Miniaturized DTM Device (MDTMD). Depiction A is a top view of the MDTMD device that shows the display unit. B shows the side view of the thin plastic ring close to the finger tip that mounts the skin temperature sensor. In an alternative embodiment, the temperature sensor is disposed in a stretch tube-shape (sleeve) over the finger, for example from the base of the finger to near the tip or last inter phalangeal crease. This embodiment may be preferred where the fingertip is needed for sensory controlled functions of the finger.

Depiction C of FIG. 4, shows the cable connecting the skin temperature sensor and the occluding band, while D shows a close up view of the MDTMD. Depiction E is an end-on view. A strap connects the two rings that lock themselves when the top ring is twisted. The strap is also to ensure a snug and comfortable fit. In F, a button on the stationary ring is to deploy the deflation process ensuring that two rings come back to their original position. G depicts another projection illustrating the MDTMD. The device is dimensioned not to interfere with normal subject prehensile or ambulatory function, and will work by triggering reactive hyperemia followed by temperature measurement using micro-transducers.

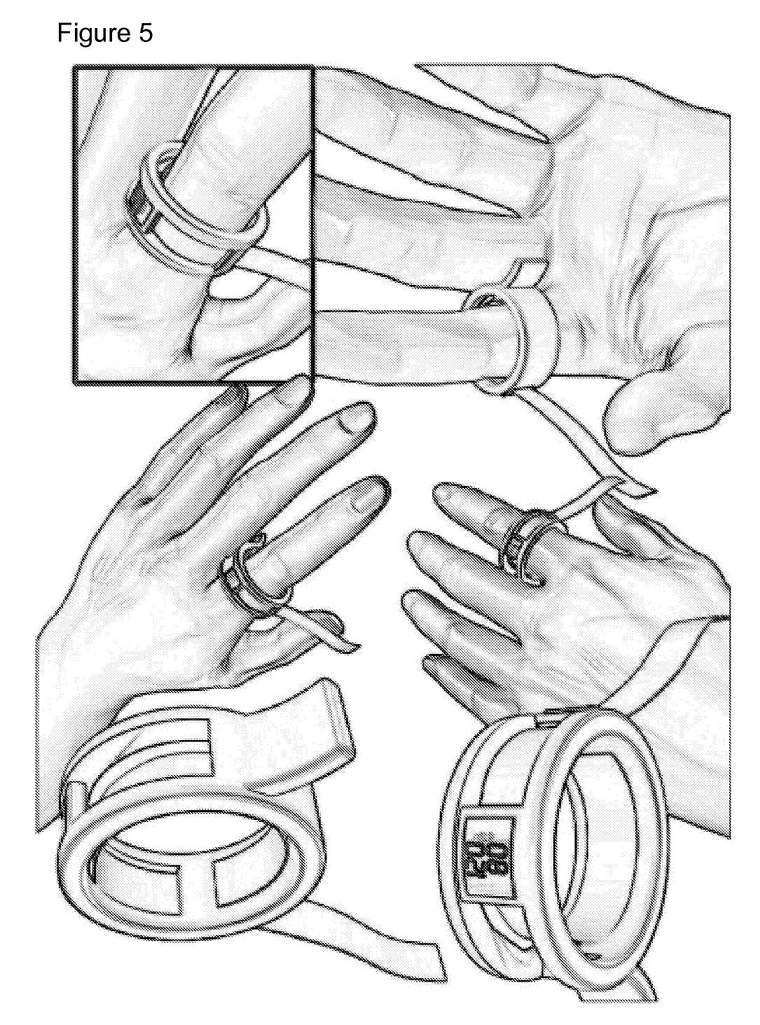

In another embodiment of a finger cuff ring is depicted in FIG. 5, the occlusion is manually implemented by pulling a strap. The ring includes a strap stop as well as a quick release mechanism that releases the tension on the strap by pressing a button on the ring. In other embodiments, the strap release is implemented by further tugging on the strap to disengage the stop mechanism. As depicted in FIG. 5, a read-out of blood pressure can be optionally displayed on the ring.

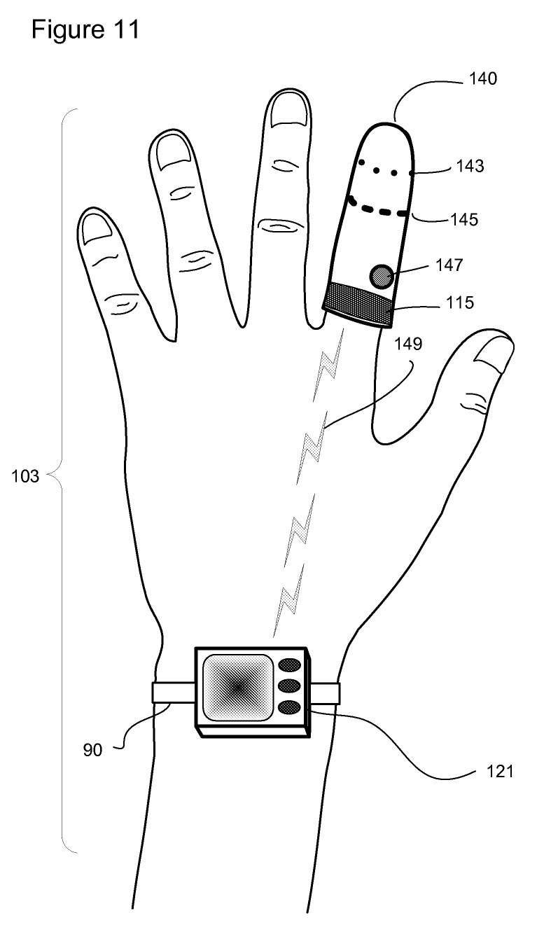

In one embodiment of the invention, an example of which is depicted in FIG. 11, the finger temperature sensor 143 is disposed within in a finger cap 140 that generally isolates the finger from rapid changes in ambient temperature such that finger temperature is principally modulated by the circulation without interference from environmental conditions. In another embodiment, the cap includes a sweat sensor 145 disposed within the cap and in skin contact. Sweating is also controlled by ANS and is another measure of a mental stress response. The cap can be alternatively connected to a light watch-type system on the wrist or a ring-like sensor (detector) system on the base of the finger or it can have a wireless micro/nano fabricated transducer that would eliminate the need for wiring. The cap can optionally be provided with a surface sensor 147 for monitoring ambient temperature. Monitoring ambient temperature, local skin temperature, and/or remote body temperature (e.g. chest or abdomen) can enable more accurate monitoring of ANS activity and mental stress. Signals 149 from each of the sensors to a controller 121 are electrically conveyed via any suitable wireless technology, or alternatively by one or more wires. In an embodiment, signals 149 can be electrically conveyed via any suitable technology from the sensors and controller 121 to outside devices and services to provide additional remote monitoring capabilities.

FIG. 6 depicts an embodiment of a hand mounted control unit that may include one or more of telemetry receiver, telemetry transmitter, data storage, battery power, digital or analog display, control buttons, timers, ambient temperature sensor, galvanic skin response (i.e., electro dermal response-EDR) monitor and a pump for controlled inflation of a finger cuff.

In one embodiment, the ring including an occluding strap or cuff also contains an additional temperature sensor that measures the ambient temperature. Both these temperature signals are digitized by a microchip-based data acquisition system, placed within the temperature sensing band (TSB). Data is recorded for a pre-set programmable duration, sufficient to capture all relevant trends of the temperature data. Upon completion of the test, the MDTMD transmits the temperature data to a remote telemedic computer system. The transmitted data will also contain “envelope” information identifying the device serial number, thereby identifying the human subject; several hundred simultaneous data transmissions can be handled by a dedicated telemedic computer system.

At the telemedic center, the dedicated computer system analyzes the temperature trends, and looks up relevant patient-specific information from its database. Using these inputs, a computational model calculates the DTM indices describing the functioning of the endothelial system. Physicians will thus be able to query and view various graphs and data tables and analyze the DTM indices to determine the patient's state of health. Having simultaneous access to the patient's medical history, they will be able to compare current data with past data taken under user-selectable environments. This will further allow the medical staff to take into account the various subjective environmental factors before arriving at a diagnosis. Table 1 below summarizes the salient features of this embodiment of a MDTMD according to the invention:

TABLE 1
Features of MDTMD

| | |
|---|---|
| 1 | Disposable temperature sensor probes. |
| 2 | Small and ergonomically designed device to allow for normal |
| | use of hands. |
| 3 | Use of biocompatible materials and adhesives. |
| 4 | High data storage capacity. Can store up to one week of continuous |
| | data feed. |
| 5 | Efficient wireless data transfer and management. |
| 6 | Impact proof. |
| 7 | Easy to use and removable and can be dismantled. |

Using a prototype device, it was demonstrated that finger based cuff occlusion yields the same results as the arm occlusion. By employing DTM in conjunction with reactive hyperemia in normal activities versus in mental stress inducing activity, individual responses to mental stress as reflected in vascular function can be determined. Further, one may also thereby determine—indirectly—the effects of mental stress on diseases such as CVD and diabetes, which have been shown to be correlated with mental stress.

In other embodiments of the present invention, finger based cuff occlusion is utilized with other methods of measuring vascular reactivity including fingertip arterial tonometry (for example using a device available from Itamar Medical) or heat-flux metering (for example using a perfusion device by Hemedex Inc).

Military Indications: Recent research has established that combat, exposure to heavy casualties, deployment of units in a war zone, and unexpected mobilizations of reserve units are all correlated with higher levels of psychological mental stress. Clinically silent coronary artery disease is well documented in apparently healthy military personnel, many of whom are routinely and chronically exposed to episodic psychological mental stress at levels that may promote CVD. Because mental stress impairs mental acuity, awareness, perception, and decision-making ability, mental stress is a potential risk factor not only for the individual concerned, but also for subordinates and fellow soldiers in combat situations. Effects of mental stress may manifest suddenly and unexpectedly, as in the case of acute coronary syndromes and sudden cardiovascular death in combat-related circumstances. Indeed, heart attack and mental stress-related illness are leading causes of death and disability, second only to combat casualties. See Enos W F et al. “Coronary Disease Among United States Soldiers Killed in Action in Korea: Preliminary Report” Journal of the American Medical Association 152 (1953) pp. 1090-1093.

Considerable attention has thus been paid to the relationship between combat and the emotional health of military personnel, including in the prevention of post-traumatic mental stress disorder. The effects of chronic mental stress impose an enormous burden on Veterans' health services. In a study conducted on 187 veterans, referred through a Veterans Administration hospital, 100 were confirmed as meeting the DSM-III criteria for PTSD. Nineteen of the 100 veterans had made a post-service suicide attempt, and 15 more had been preoccupied with suicide since the war. Five factors were significantly related to suicide attempts: guilt about combat actions, survivor guilt, depression, anxiety, and severe PTSD. See Hendin H and Haas A P “Suicide and guilt as manifestations of PTSD in Vietnam combat veterans” Am J Psychiatry 148 (1991) 586-591.

The more dramatic aspects of wartime activities have been clearly established as precipitants of psychological mental stress. However, at present there are no practical means to continuously monitor the cardiovascular effects of mental stress in ambulant subjects. The magnitude of the mental stress burden of combat conditions is therefore unknown, as is the extent of variation in individual susceptibilities. Thus it is very difficult to predict which individuals will come to harm from repeated exposure to mental stress. Although heart rate and blood pressure monitoring can provide crude indicators of mental stress and vascular function, the latter is impractical in combat.

Due to a lack of rigorous and sensitive methods of measuring the impact of the psychological factors on the cardiovascular system, the insidious and slowly developing symptoms of mental stress often go unrecognized and the effects of mental stress are often only recognized subsequent to severe trauma or functional disruption of the patient. See Blood C G and Gaucher E D “The relationship between battle intensity and disease rate among Marine Corps infantry units” Milit Med 158 (1993) 340-4. Treatment strategies commonly involve drastic life-style changes or heavy medication; there is no other recourse given the acuteness of the disease.

In a military context, seemingly healthy military personnel may also be at considerable risk of cardiovascular events, particularly in mission-critical situations with high levels of physical and psychological mental stress. At present, the cardiovascular fitness levels sufficient to tolerate mental stress, and the adverse short- and long-term cardiovascular effects of mental stress cannot be quantified. Conventional clinical assessment of cardiovascular (CV) fitness in apparently healthy subjects, such as active duty military personnel (e.g. screening for CV risk factors, exercise mental stress testing), fails to identify individuals with occult coronary heart disease (CHD), who are at increased near-term risk of cardiovascular events. The routine use of coronary imaging technologies (such as computer tomography, CT, heart scanning) to screen for silent CHD is cost-prohibitive, particularly in relatively young subjects.

It is clear that the investigation of psycho-social impacts is vital, especially in military contexts where mental stress reactions can be widespread and acute. The present invention is adapted to (a) identifying individual-specific susceptibilities to mental stress, (b) quantifying the risk factors, and (c) promoting early identification and thus effective prevention and rehabilitation of pathologic mental stress responses. The present invention provides methods and apparatus for ambulatory monitoring of mental stress response as manifest by cooling of the skin temperature. In addition, vascular reactivity can be assessed by implementing reactive hyperemia in ambulatory individuals as implemented using a finger-mounted arterial occlusion cuff.

In one embodiment of the present invention, mental stress monitoring by continuous skin temperature recording is combined with real and induced mental stress situation to provide individual assessments of mental stress responses. In another embodiment, vascular reactivity is assessed during real-life activities by utilizing combined finger and palm temperature monitoring. In another embodiment, finger based cuff occlusion of the present invention implements reactive hyperemia to measure vascular reactivity by DTM and/or fingertip arterial tonometry. The device is worn in ordinary conditions, normally considered to be non-mental stressful, to establish a “normal” individual vascular reactivity profile. The individual is then subject to various mental stressful conditions to determine that individual's vascular reactivity under mental stress.

In one embodiment, the mental stress is induced by a virtual reality combat situation simulator. One example of a combat-level mental stress simulator is the Immersive Virtual Reality (IVR) high-definition, use-of-force firearms simulator system developed by VirTra Systems Inc. The simulator—originally designed as a training system—creates environments mimicking real-life combat situations using advanced projection technologies. The fear of injury can also be simulated using VirTra Systems' Threat-Fire™ belt, which permits an instructor to deliver an electric “stun” to a trainee, simulating the sensation of being shot, thus realistically simulating the real mental stress associated with such a situation. The simulator affords a variety of scenarios, ranging from military “fourth-generation” combat to urban law enforcement.

Use of mental stress simulators in conjunction with ambulatory temperature recording as a monitor of an individual mental stress response confers several benefits. First, it allows standardization of mental stress conditions. Second, a series of controlled experiments with varying degrees of mental stress under repeatable conditions can be simulated, thereby facilitating precise measurements. Third, potentially significant variations in factors such as weather conditions, food intake and other conditions that could lead to increased measurement noise can be avoided. Use of IVR systems permits isolation of physiological mental stress from that of the mental stress experience due to physical exertion. Individuals who are more susceptible to mental stress can be readily identified. In a further embodiment, mental stress monitoring by continuous skin temperature recording is combined with ambulatory vascular response monitoring using finger cuff occlusion to stimulate reactive hyperemia to identify those individuals who react dangerously to mental stress and to quantify relative responses. In another embodiment, mental stress monitoring by continuous skin temperature recording and/or ambulatory vascular response monitoring is further utilized to teach control of potentially dangerous mental stress and vascular responses.

In one embodiment of the invention, ambulatory mental stress response monitors and/or vascular response monitors are combined with mental stress inducers to monitor correlations between emotional mental stress and current or future cardiovascular health, as conventionally measured in the art (e.g. using treadmills with EKG). Such correlations provide for improved screening and monitoring methods for military personnel, and facilitate early diagnosis and treatment as well as provide a metric for developing and implementing remedial or preventive treatments.

In one embodiment of the invention, ambulatory mental stress and/or vascular response monitors are combined with mental stress inducers to differentiate the subjects most susceptible to mental stress (most acutely manifested in military environments) from other mentally and physically healthy individuals. The objective data provided by the present invention enables development and implementation of remedial or preventive treatments for mental stress-susceptible individuals in mission critical situations. Remedial action taken in early stages of CVD has been clearly shown to cause disease regression and improved CV health.

In one embodiment, the ambulatory mental stress and/or vascular response monitors of the present invention are continuously worn by soldiers to enable not only the gathering of data during periods of mental stress, but also in the long term aid in better health management and deployment of medical aid. Advances in information technologies enable rapid data retrieval and electronic communications in all aspects of military operation. In particular, technologies that facilitate medical force management using telemedics and advanced diagnostics complement the existing resources of modern highly mobile and remotely deployed armed forces. The telemedic computer system will also be potentially capable of transmitting any medical prescriptions or advice to the communication systems carried by the military personnel in the field. The deployment of the MDTMD fits well with the military's evolving philosophy of how technology can aid the diagnosis of medical conditions and their effective and efficient treatment through telemedics. In one embodiment of the present invention, finger based cuff occlusion is utilized with methods of measuring vascular perfusion including DTM and fingertip arterial tonometry (for example using a device available from Itamar Medical). The device is worn in ordinary conditions, normally considered to be non-mental stressful, to establish a “normal” individual vascular reactivity profile. The individual is then subject to various mental stressful conditions to determine that individual's vascular reactivity under mental stress. In one embodiment, the mental stress is induced by a virtual reality combat situation simulator.

Use of Ambulatory Mental stress and Vascular Response Monitors in Conjunction with Risk Factor Assessment: Mental stress may manifest itself in different ways in healthy young military soldiers as compared to the older war veterans or high rank officers as well as civilians. Among sensitive individuals, the presence of inflammatory markers that promote CHD could contribute to a condition in which the slightest of the triggers due to psychological mental stress can be fatal. Military personnel and civilians alike include individuals who have subclinical atherosclerosis as measured by coronary artery calcium score (CACS) and carotid intima media thickness (CIMT). Certain of these individuals are more susceptible than others to psychological mental stress that is manifest in sympathetic nervous system vasoconstriction that is potentially life threatening. These are the individuals who are on the “fast-track” to CHD. On the other hand, the effect of mental stress on younger soldiers and civilians could be slightly different and will have a long-term effect on vascular health. Therefore, there is a need to identify those individuals that are classified as Very-High-Risk according to further criteria including for example those put forth in the SHAPE Task Force guidelines. See Naghavi M et al. “From Vulnerable Plaque to Vulnerable Patient: A Call for New Definitions and Risk Assessment Strategies Part I” Circulation 108 (2003) 1664-1672; Naghavi M et al. “From Vulnerable Plaque to Vulnerable Patient: A Call for New Definitions and Risk Assessment Strategies: Part II.” Circulation 108 (2003) 1772-1778.

Use of Ambulatory Mental stress and Vascular Response Monitors in Secondary Prevention: In one embodiment of the invention, the ambulatory mental stress and/or vascular response monitors described herein are implemented for use in heart attack patients after they are released from hospital. Certain of these individuals are more susceptible than others to detrimental effects of psychological mental stress that increase their risk of recurrent (future) adverse events. It is believed that individuals with psychological mental stress such as mental depression and anxiety have a 4-5 times increased risk of a second heart attack. The present invention provides means to identify these individuals. Thus, in one embodiment of the present invention, ambulatory mental stress monitoring by continuous skin temperature measurement is employed to identify dangerous mental stress responses and thus provides a mechanism to identify these susceptible individuals as prevention modality for a secondary heart attack.

In one embodiment, the software or algorithms governing such ambulatory (or non-ambulatory) monitoring device can include other useful information related to psychological status of the patient (e.g. questions from standard depression evaluation questionnaires) to complement the diagnostic value of the device. In another embodiment, ambulatory mental stress monitoring by continuous skin temperature measurement is employed as an objective biofeedback reporter to teach control of mental stress responses. In one embodiment, the device can have (or be accompanied or coupled with) biochemical sensors for measurement of biomarkers of mental stress (such as saliva cortisol) to complement the diagnostic accuracy of the monitoring device (system). In another embodiment, ambulatory mental stress monitoring by continuous skin temperature measurement is employed to monitor the effects of intervention strategies as an objective reporter of the success of therapies for mental stress control including by pharmacologic interventions.

We claim:

1. An ambulatory method for profiling an individual's vulnerability to detrimental effects of mental stress on vascular function comprising:
monitoring a baseline of mental stress levels and a baseline of vascular function levels in the individual;
monitoring changes in the mental stress levels in the individual during a mental stress challenge;
monitoring changes in the vascular function levels in the individual during the mental stress challenge; and
correlating the changes of mental stress levels with the changes of vascular function levels to profile the individual's vulnerability to detrimental effects of mental stress on vascular function.

2. The ambulatory method of claim 1, wherein the monitoring of baseline and changes of mental stress levels is a measurement selected from the group consisting of: fingertip temperature, saliva cortisol, fingertip pressure, tissue perfusion, sweating, pulse rate, blood pressure, galvanic response, microneurography, or combinations thereof.

3. The ambulatory method of claim 1, wherein the monitoring of baseline and changes of vascular function levels is determined by a method selected from the group consisting of: Digital Thermal Monitoring, tonometry, Doppler ultrasound, laser Doppler flowmetry, photoplethysmography, iontophoresis, measuring changes in magnetic or electromagnetic properties of the tissue, or combinations thereof.

4. The ambulatory method of claim 1, further comprising determining the cardiovascular risk factors of the individual.

5. An ambulatory device capable of monitoring an indicator of mental stress and an indicator of vascular function.

6. The ambulatory device of claim 5, wherein the ambulatory device is worn on a wrist, is in communication with at least one finger mounted sensor, is capable of inducing a reactive hyperemia response distal to the device, and contains at least one display.

7. The ambulatory device of claim 5, wherein the indicator of mental stress is a measurement of blood flow to an extremity such as fingertips.

8. The ambulatory device of claim 5, wherein the indicator of mental stress is selected from the group consisting of: fingertip temperature, saliva cortisol, fingertip pressure, tissue perfusion, sweating, pulse rate, blood pressure, galvanic response, microneurographs, or combinations thereof.

9. The ambulatory device of claim 5 wherein the indicator of vascular function is a measurement of reactive hyperemia.

10. The ambulatory device of claim 5, wherein the indicator of vascular function is measured by a method selected from the group consisting of: Digital Thermal Monitoring, tonometry, Doppler ultrasound, laser Doppler flowmetry, photoplethysmography, iontophoresis, measuring changes in magnetic or electromagnetic properties of the tissue, or combinations thereof.

11. An ambulatory device for measuring mental stress comprising:
at least one finger mounted blood flow monitor in electrical communication with a control unit; and
at least one palm mounted blood flow monitor in electrical communication with the control unit, wherein the control unit is adapted to continuously measure and record data from both finger and palm mounted blood flow monitors.

12. The ambulatory device of claim 11, wherein the finger and palm mounted blood flow monitors are selected from the group consisting of: inherent temperature sensors, induced temperature sensors, tonometry sensors, laser Doppler flowmetry sensors, and photoplethysmography sensors.

13. The ambulatory device of claim 11, further comprising an ambient temperature sensor.

14. The ambulatory device of claim 11, further comprising a controller in electrical connection to the blood flow monitors.

15. The ambulatory device of claim 11, further comprising an occlusion cuff for inducing a reactive hyperemia response.

16. The ambulatory device of claim 15, wherein the occlusion cuff is a finger mounted occlusion cuff disposed proximal to the finger mounted blood flow sensor.

17. The ambulatory device of claim 15, wherein the occlusion cuff is a wrist mounted occlusion cuff disposed proximal to the finger and palm mounted blood flow sensors.

18. The ambulatory device of claim 11, further comprising an additional physiologic parameter monitor selected from the group consisting of monitors for: pulse rate, blood pressure, galvanic response, sweating, blood oxygenation, core temperature, and/or skin temperature on the thoracic or truncal (abdominal) part.

19. The ambulatory device of claim 11 wherein the ambulatory device is disposed within a glove.

20. An ambulatory device for measuring reactive hyperemia comprising:
a ring dimensioned to be worn on a finger, the ring including a band that can be controllably tightened on the finger thereby restricting an arterial flow distal to the ring;
a release for relieving the restriction of the band thereby restoring arterial flow distal to the ring; and
a sensor for measuring blood flow to a fingertip distal to the cuff.

21. The ambulatory device of claim 20 wherein the ring is inflatable.

22. The ambulatory device of claim 20, wherein the band is a strap that is tightened manually.

23. The ambulatory device of claim 20, wherein the sensor can be selected from a group consisting of: a temperature sensor, a fingertip arterial tonometry sensor, or combinations thereof.

24. The ambulatory device of claim 20, further comprising an ambient temperature sensor.

25. The ambulatory device of claim 20, wherein the ambulatory device is disposed in a glove.
